(19) United States
(12) Patent Application Publication
(10) Pub. No.: US 2012/0184033 A1
(43) Pub. Date: Jul. 19, 2012

(54) CONTAINER FOR STORING A FLOWABLE BODILY MATERIAL, AND A METHOD FOR STORING A FLOWABLE BODILY MATERIAL

(75) Inventors: David John Crimmins, Swords (IE); Margaret Mary Goggin, Castieknock (IE)

(73) Assignee: FUIL TECHNOLOGIES LIMITED, Dublin (IE)

(21) Appl. No.: 13/265,284

(22) PCT Filed: Apr. 20, 2010
(86) PCT No.: PCT/IE2010/000027

§ 371 Date: Apr. 6, 2012

(30) Foreign Application Priority Data
Apr. 20, 2009 (IE) S2009/0309

(51) Int. Cl. C12N 5/02; C12M 1/00

(52) U.S. Cl. 435/374; 435/307.1

(57) ABSTRACT

A container (1) for storing stem cell material of an animal for subsequent treatment of the animal contains discrete pouches (15) for the stem cell material. The container (1) comprises a pair of transparent flexible panels (3) which are secured together by a peripheral heat seal (4) to define a hollow interior region (8). The hollow interior region (8) is divided into discrete pouches (15) and a mixing region (16) by primary heat seals (18) extending substantially perpendicularly from the peripheral seal (4) adjacent an edge (5a) of the panels (3). An inlet port (9) to the mixing region (16) accommodates the stem cell material and a protective solution to be mixed in the mixing region (18). A manifold (25) extending transversely of the pouches (15) communicates the pouches (15) with the mixing region (16). On completion of mixing of the stem cell material with the protective solution, two additional pouches (22) are formed in the mixing area (16) by primary heat seals (34). The mixed material is then urged along the manifold (25) into the pouches (15,22), and a secondary heat seal (39) isolates the discrete pouches (15,22) from the manifold (25) and from each other to thereby seal the mixed material in the pouches (15,22). On the formation of the secondary heat seal (39) the sealed pouches (15,22) are severed from the container (1) along the cut lines (17,36,42) to be separately stored. Sealed outlet ports (20) from the respective pouches (15,22) through the peripheral seal (4) adjacent the edge (5a) of the panels (3) facilitate withdrawal of the stem cell materials from the respective pouches (15,22).

The present invention relates to a container for storing a flowable bodily material of a human or animal, and in particular, though not limited to a container for cryogenically storing stem cells extracted from blood serum. The invention also relates to a method for storing a flowable bodily material of a human or animal, and in particular, though not limited to a method for cryogenically storing stem cells from blood.

It has been found that the results achieved when treating injuries to animals, as well as treating inflammation and other ailments in animals can be significantly enhanced by cell therapy, whereby stem cell material is applied to the affected site of the animal. Ideally, the stem cell therapy should be autologous stem cell therapy, whereby the stem cells are harvested from the animal being treated, as opposed to allogeneic stem cell therapy, whereby the stem cells are harvested from another animal. Additionally, it is important that the stem cells be of the type which readily convert to the tissue or organ being treated. It is known that the most adaptable stem cells are those which are harvested from blood of the placenta and the umbilical cord attached to the animal at birth. Stem cell material is harvested from the blood of the placenta and the umbilical cord by centrifuging the blood in order to separate out the stem cell material. The stem cells reside within an area populated by white blood cells (leukocytes) and is commonly referred to as buffy coat, which is a thin greyish white layer which forms between the platelets and plasma at the top and the packed red blood cells of a haematocrite at the bottom, after centrifuging.

However, in general, treatment of injuries, inflammation and other ailments of an animal, in general, do not arise for some considerable time after birth, and in many cases, many years after birth. This, thus, requires storing the stem cells which have been harvested from the placenta and the umbilical cord of the animal at birth, and also storing the harvested stem cells so that they can be readily retrieved and identified as being harvested from the placenta and the umbilical cord of the specific animal.

Such stem cells must be cryogenically stored, and typically, must be stored at temperatures in the range of −150° C. to −196° C. In general, stem cells when harvested from a placenta and the umbilical cord are placed in a container and stored cryogenically. However, a problem with these known techniques is that once the container containing the stem cells has been removed from the refrigerator or other cryogenic storing apparatus, the temperature of the container must be raised in order to retrieve the stem cells. However, since only a small number of stem cells are required for treatment, the remaining stem cells in the container in general cannot be successfully refrozen, and thus, must be destroyed. This is a considerable problem, and in general, results in only one single treatment being available to an animal.

There is therefore a need for a method and apparatus which addresses this problem.

The present invention is directed towards providing a container for storing a flowable bodily material of a human or animal, and the invention is also directed towards providing a method for storing a flowable bodily material of a human or animal.

According to the invention there is provided a method for storing a flowable bodily material from a human or animal body, the method comprising providing a container comprising a pair of panels of flexible material sealed together to define a hollow interior region by a peripheral seal extending around the hollow interior region, and an inlet port being provided to the hollow interior region, securing the panels together to define at least two discrete pouches and a manifold in the hollow interior region, with the manifold communicating the respective pouches with the inlet port, delivering the bodily material through the inlet port to the pouches through the main manifold, and further securing the panels together to isolate the pouches from the manifold and from each other with the bodily material sealed therein.

In one embodiment of the invention the panels are secured together to define a mixing region in the hollow interior region for mixing the flowable bodily material with a protective material. Advantageously, the mixing region is located adjacent the inlet port.

In another embodiment of the invention the manifold forms the mixing region.

In a further embodiment of the invention the manifold communicates with the inlet port through the mixing region.

In one embodiment of the invention the panels are secured together at a location in the mixing region to form at least one additional discrete pouch from the mixing region, the at least one additional discrete pouch communicating with the manifold.

Preferably, the bodily material and the protective material are mixed in the mixing region prior to forming the at least one additional pouch in the mixing region.

Advantageously, the panels are secured together along respective spaced apart elongated primary sealing areas to define the discrete pouches. Preferably, the primary sealing areas with the exception of the primary sealing area closest to the inlet port extend substantially parallel to each other. Ideally, the primary sealing area which is closest to the inlet port extends in a general direction towards the adjacent primary sealing area.

In one embodiment of the invention the primary sealing area which forms the at least one additional discrete pouch in the mixing region which is closest to the inlet port extends in a general direction towards the adjacent primary sealing area. Preferably, the primary sealing areas extend from the peripheral seal. Advantageously, the primary sealing areas extend from the peripheral seal substantially perpendicularly thereto.

In another embodiment of the invention the panels are secured together along the primary sealing areas by heat sealing the panels. Preferably, the primary sealing areas are of sufficient width to permit severing of the sealed pouches from the container along the primary sealing areas without affecting the integrity of the seals.

In a further embodiment of the invention the panels are secured together along secondary sealing areas for isolating the respective pouches from the manifold and from each other to sealably close the respective pouches. Preferably, the panels are secured together along the secondary sealing area by heat sealing the panels. Advantageously, the secondary sealing area is of sufficient width to permit severing of the sealed pouches from the container along the secondary sealing area without affecting the integrity of the secondary seal.

Preferably, the pouches extend side by side from the peripheral seal. Advantageously, the manifold extends transversely relative to the pouches.

In one embodiment of the invention a sealed outlet port is provided from each pouch. Preferably, each sealed outlet port is adapted to be punctured for withdrawing the bodily material therefrom. Advantageously, each sealed outlet port is adapted to be punctured by a pointed tip of a cannula. Ideally, the sealed outlet ports are located adjacent one end of the respective pouches.

In one embodiment of the invention the sealed outlet ports are located adjacent a portion of the peripheral seal which extends adjacent the edge of the panels. Preferably, each sealed outlet port is located between the panels adjacent the peripheral seal and the panels are sealably secured to the sealed outlet ports. In one embodiment of the invention the panels are sealably secured to the outlet ports by heat sealing.

In another embodiment of the invention the peripheral seal which defines the hollow interior region between the panels extends around the periphery of the panels.

In an alternative embodiment of the invention the peripheral seal which defines the hollow interior region between the panels is spaced apart from a portion of the periphery of the panels.

In another embodiment of the invention a second inlet port is provided to the hollow interior region for accommodating a protective material for mixing with the bodily material.

Preferably, each inlet port is located between the panels adjacent the peripheral seal, and is sealably secured to the panel. Advantageously, each inlet port is sealably secured to the panels by heat sealing.

Preferably, each inlet port is adapted to be sealably coupled to a corresponding supply tube from which the bodily material is supplied to the inlet port. Advantageously, an inlet tube extends outwardly from each inlet port and is adapted for sealably coupling to the corresponding supply tube. Ideally, the inlet tube of each inlet port is adapted to be sealably coupled to the corresponding supply tube by heat sealing.

In one embodiment of the invention the container is adapted for storing stem cell material extracted from blood.

The invention also provides a container for use in the method according to the invention.

Further the invention provides a container for storing a flowable bodily material of a human or animal, the container comprising a pair of panels of flexible material sealed together by a peripheral seal to define a hollow interior region, and an inlet port being provided to the hollow interior region, the panels being one of secured together to define at least two discrete pouches and a manifold and adapted to be secured together to define the at least two discrete pouches and the manifold in the hollow interior region with the manifold communicating the respective pouches with the inlet port.

In one embodiment of the invention the panels are one of secured together to define a mixing region and adapted to be secured together to define the mixing region in the hollow interior region for mixing the flowable bodily material with a protective material.

In another embodiment of the invention the manifold forms the mixing region.

In another embodiment of the invention the panels are adapted to be secured together at a location within the mixing region to form at least one additional discrete pouch in the mixing region communicating with the manifold.

Preferably, each pouch is an elongated pouch. Advantageously, the pouches extend side by side from the peripheral seal. Ideally, the manifold extends transversely relative to the pouches.

In one embodiment of the invention the primary sealing area which separates the mixing region from the adjacent discrete pouch extends in a general direction towards the adjacent primary sealing area.

In another embodiment of the invention the panels are adapted to be secured together along secondary sealing areas for isolating the respective pouches from the manifold and from each other to sealably close the respective pouches.

Preferably, the container is one of provided with a plurality of sealed outlet ports and adapted to be provided with the plurality of sealed outlet ports, such that one of the sealed outlet ports is provided from each pouch.

Preferably, the outlet ports are located between the panels adjacent the peripheral seal. Advantageously, the panels are sealably secured to the outlet ports. Ideally, the panels are sealably secured to the outlet ports by heat sealing.

In one embodiment of the invention the material of the panels of the container is a material resistant to cryogenic temperatures. Preferably, the material of the panels of the container is of a material which does not react with the bodily material. Advantageously, the material of the panels of the container is a fluorinated ethylene-propylene (FEP) plastics material.

In one embodiment of the invention the container is adapted for storing stem cell material extracted from blood.

The advantages of the invention are many. A particularly important advantage of the invention is that relatively large quantities of stem cells can be harvested from the blood of the placenta and the umbilical cord of an animal at birth and can be stored in relatively small quantities in the discrete pouches which may be sized to be suitable for a single treatment of the animal and which are independently retrievable. By virtue of the fact that the stem cells are stored in a plurality of relatively small pouches, the pouches can be retrieved from a refrigerator or other cryogenic apparatus independently of each other as required, thus avoiding wastage of stem cells, and providing many treatments of stem cells for the animal. Additionally, since the samples are stored in separate discrete pouches, the pouches may be stored in respective different cryogenic units, which may be at different geographical locations, thereby reducing the risk of loss or damage of all the stored samples, since the failure of a single cryogenic unit will not result in the loss of all the samples, but only those samples which are stored in the failed cryogenic unit.

A further advantage of the invention is that the method and container according to the invention permit the mixing of the stem cell material with the protective material, and the subsequent storing of the mixed stem cell material and protective material in the discrete pouches to be carried out without the need for a clean room environment.

The provision of the container of fluorinated ethylene-propylene plastics material provides a further advantage, in that the stem cell material does not adhere to the panels of the container, and thus mixing of the stem cell material with the protective material and the urging of the mixed stem cell material and the protective material into the discrete pouches can be carried out without any difficulty, and furthermore, extraction of the mixed stem cell material and protective material from the pouches subsequently can be carried out without loss of the stem cell material. Additionally, it has been found that fluorinated ethylene-propylene plastics material is a more robust material than materials used heretofore to store such samples.

The invention will be more clearly understood from the following description of some preferred embodiments thereof, which are given by way of example only, with reference to the accompanying drawings, in which:

FIG. 1 is a top plan view of a container according to the invention,

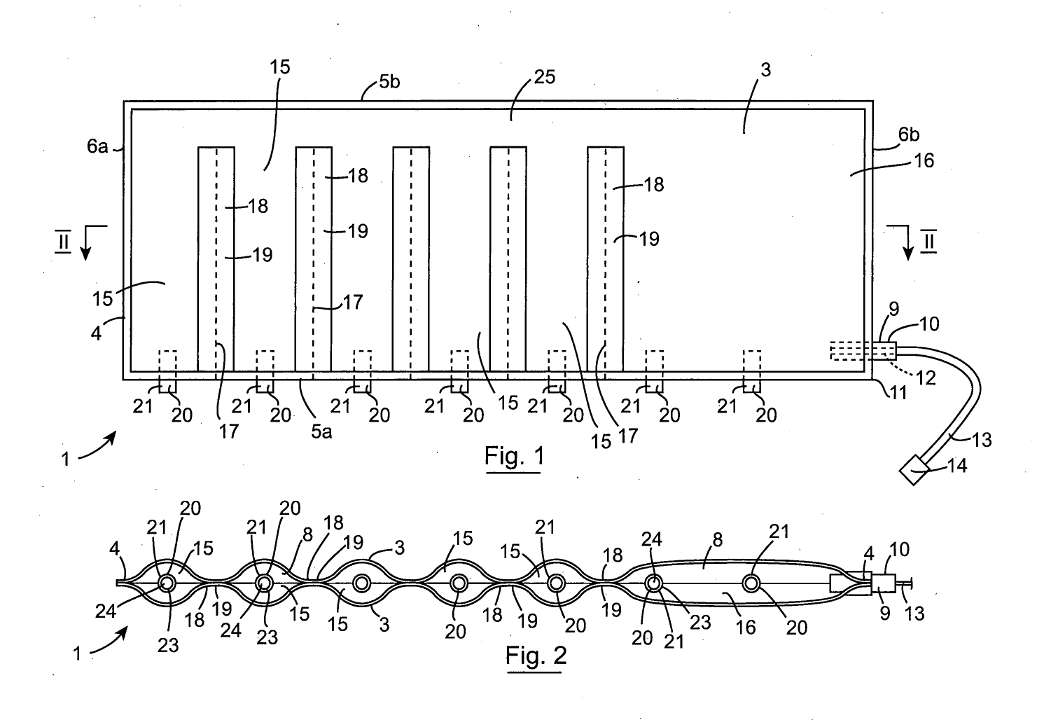

FIG. 2 is a transverse cross-sectional side elevational view of the container of FIG. 1 on the line II-II of FIG. 1,

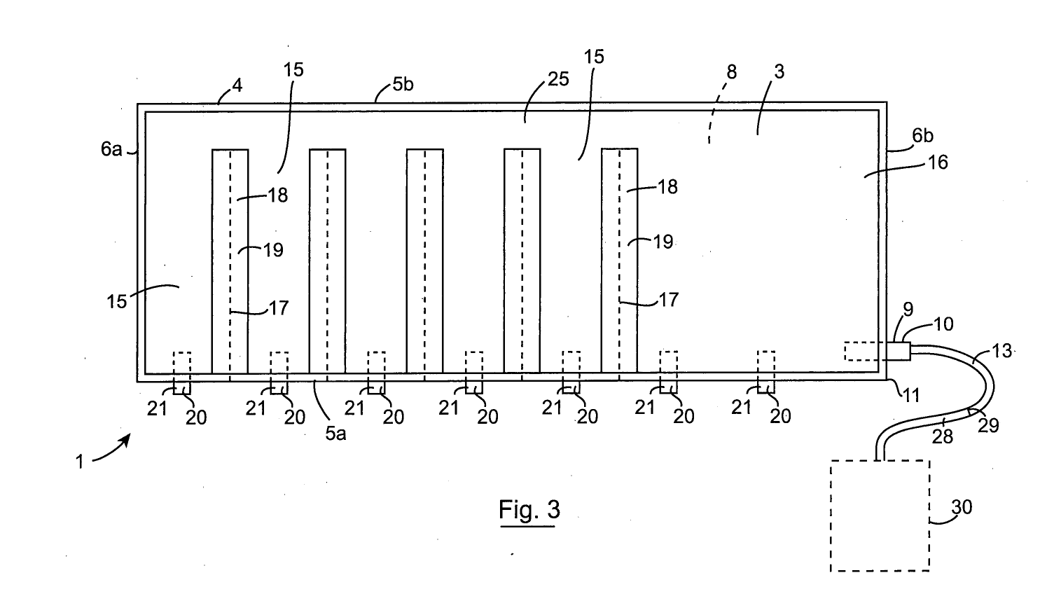

FIG. 3 is a top plan view of the container of FIG. 1 in use,

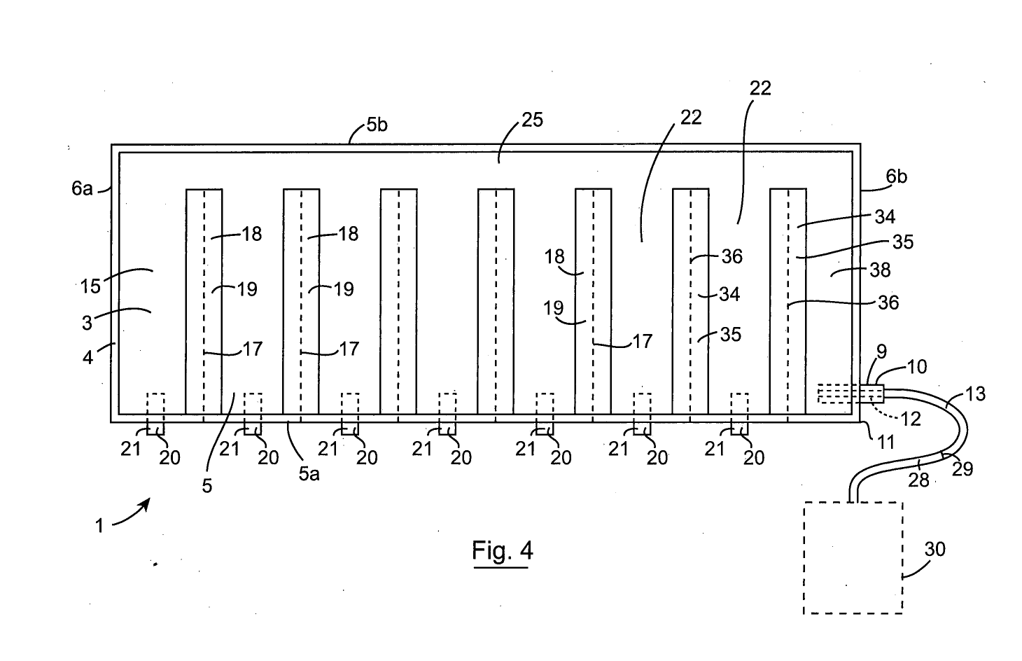

FIG. 4 is a top plan view of the container of FIG. 1 also in use,

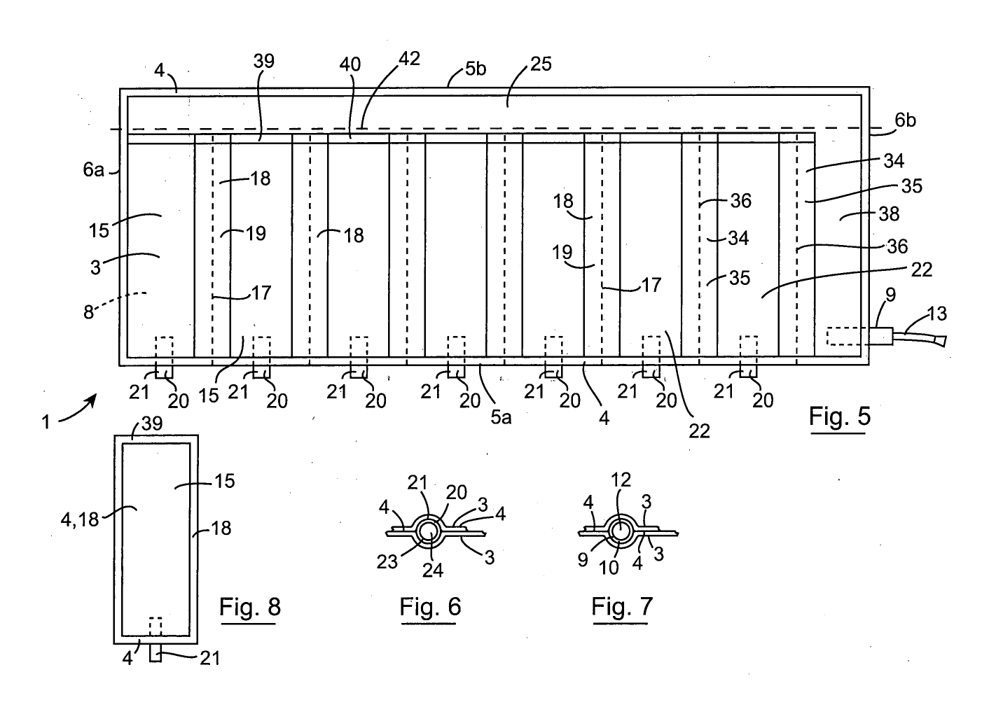

FIG. 5 is a top plan view of the container of FIG. 1 also in use,

FIG. 6 is a side elevational view of a detail of the container of FIG. 1,

FIG. 7 is an end elevational view of another detail of the container of FIG. 1,

FIG. 8 is a top plan view of a sealed pouch severed from the container of FIG. 1,

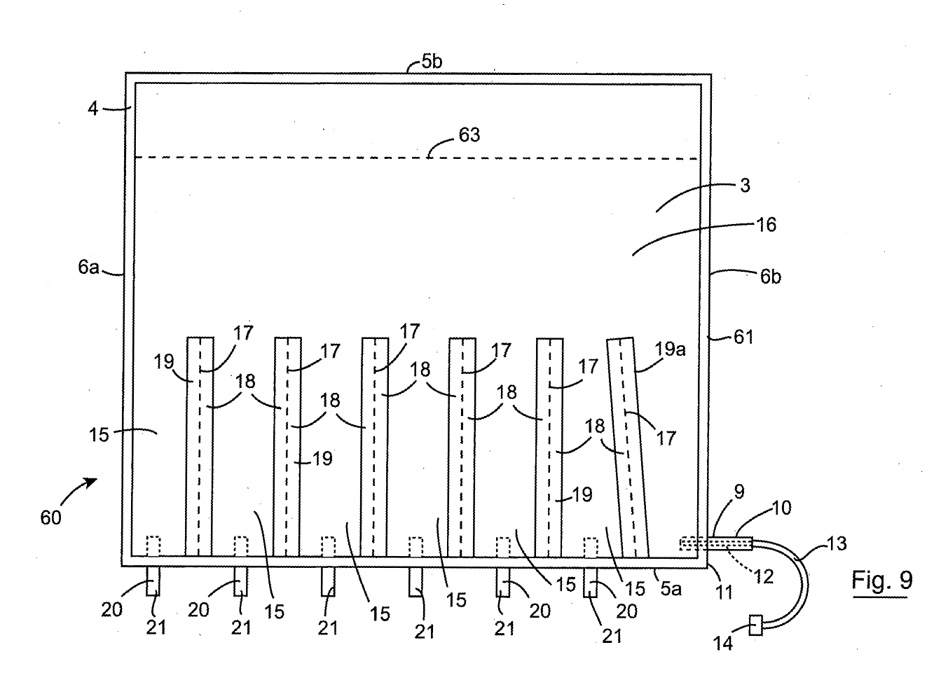

FIG. 9 is a view similar to FIG. 1 of a container according to another embodiment of the invention, and

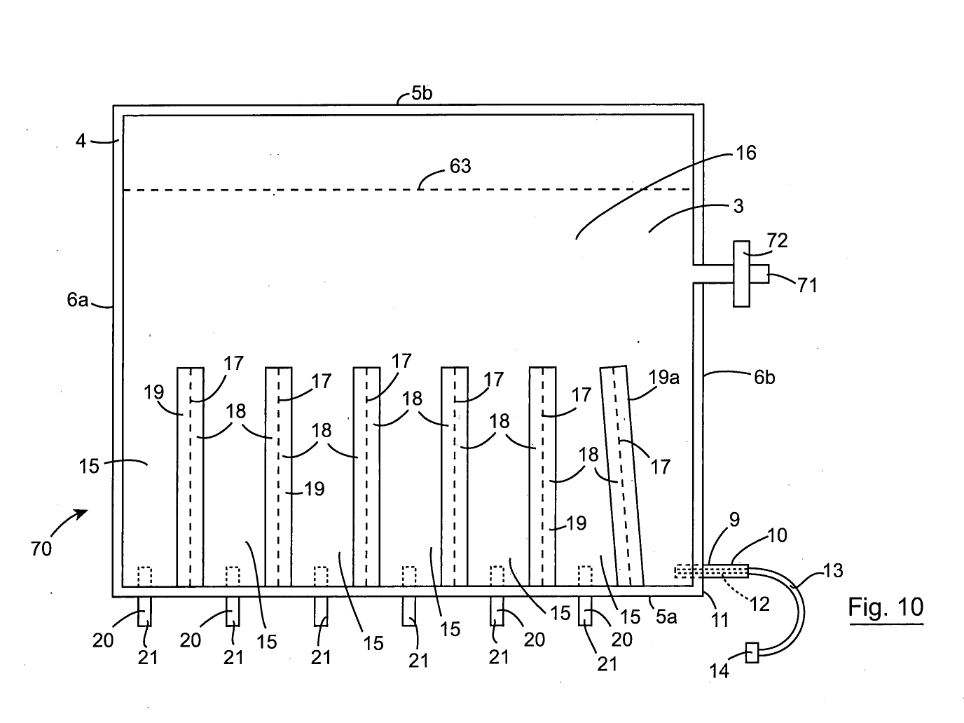

FIG. 10 is a view similar to FIG. 1 of a container according to a further embodiment of the invention.

Referring to the drawings, and initially to FIGS. 1 to 8, there is illustrated a container according to the invention, indicated generally by the reference numeral 1, for cryogenically storing a flowable bodily material of a human or animal, which in this embodiment of the invention is stem cell material, typically referred to as a buffy coat. Buffy coat is a thin greyish white layer which forms between the platelets and plasma at the top and the packed red blood cells of a haematocrite at the bottom, after centrifuging. The stem cell material may be extracted from blood of an animal, for example, a horse. In particular, the container 1, as will be described in detail below, is suitable for storing small discrete volumes of stem cell material harvested from the placenta and the umbilical cord after the birth of a foal so that the small discrete volumes of the stem cell material can be frozen at cryogenic temperatures in order to preserve the stem cells for subsequent treatment of injuries, inflammations, diseases, ailments and other episodes which may occur subsequently in the life of the horse. By treating injuries, diseases and other episodes of a horse with stem cells extracted from blood of the umbilical cord or the placenta at birth, the likelihood of an adverse autoimmune response is minimised, and in many cases eliminated, since the treatment is an autologous stem cell therapy.

The container 1 comprises a pair of panels 3 of transparent heat sealable flexible plastics material, which in this embodiment of the invention is FEP plastics material, which is resistant to cryogenic temperatures and does not react to the stem cell material. The panels 3 are secured together by a peripheral heat seal 4 which in this embodiment of the invention extends around the periphery of the container 1 along respective opposite side edges 5a and 5b and respective opposite end edges 6a and 6b. The peripheral seal 4 defines with the panels 3 a hollow interior region 8.

An inlet port 9 formed by a tubular inlet plug 10 of plastics material is located between the panels 3 adjacent a corner 11 thereof for accommodating the stem cell material into the hollow interior region 8. The inlet plug 10 of the inlet port 9 is heat sealed to the panels 3 so that the heat sealing of the inlet plug 10 to the panels 3 forms a continuous seal with the peripheral seal 4, thereby sealing the hollow interior region 8. An inlet bore 12 extends through the inlet plug 10 for communicating with the hollow interior region 8. An inlet tube 13 extending outwardly from the inlet port 9 and communicating with the hollow interior region 8 of the container 1 through the inlet bore 12 terminates in a sealed closure plug 14 for maintaining the hollow interior region 8 of the container 1 sealed. The inlet tube 13 is of a plastics material which is suitable for heat welding to a supply tube through which the stem cell material separated from the blood plasma is delivered into the hollow interior region 8 through the inlet tube 13 and the inlet bore 12 of the inlet port 9, as will be described below.

A plurality of pouches 15 and a mixing region 16 are formed in the hollow interior region 8 by heat sealing the panels 3 together along elongated primary heat seals 18 in primary heat seal areas 19 which extend perpendicularly from the portion of the peripheral seal 4 which extends along the side edge 5a of the container 1. In this embodiment of the invention five pouches 15 are formed in the hollow interior region 8 by the primary heat seals 18 of the heat seal areas 19 for storing the stem cell material.

The mixing region 16 which is defined between the portion of the peripheral seal 4 which extends along the end edge 6b and the adjacent-most primary heat seal 18 is provided for mixing the stem cell material with a protective solution prior to urging the mixed stem cell material and protective solution into the pouches 15. In this embodiment of the invention the protective solution is a custom made dimethyl sulfoxide (DMSO) saline solution for protecting the stem cell material against the cryogenic temperatures. The width of the primary heat seal areas 19 is such as to permit subsequent severing of the sealed pouches 15 from the container 1 along cut lines 17 without affecting the integrity of the primary heat seals 18.

A plurality of sealed outlet ports 20 comprising tubular outlet plugs 21 are located at spaced apart intervals along the side edge 5a of the container 1 and extend between the panels 3 to communicate with the respective pouches 15 and the mixing region 16. The outlet ports 20 are provided to facilitate subsequent withdrawal of the mixed stem cell material and the protective solution from the pouches 15. Two of the outlet ports 20 which extend from the mixing region 16 are provided for two additional pouches 22 which are subsequently formed in the mixing region 16 after the stem cell and the protective solution have been mixed therein, as will be described below. The outlet plugs 21 of the outlet ports 20 are sealed to the panels 3, so that the seal between the panels 3 and the outlet plugs 21 forms a continuous seal with the peripheral seal 4, thereby sealing the hollow interior region 8. A bore 23 extends through each outlet plug 21 and is sealed by a membrane 24 extending across the bore 23 for sealing the hollow interior region 8 and the pouches 15. The membrane 24 is of sufficient thickness to prevent accidental puncturing of the membrane 24, while at the same time permitting puncturing by a pointed tip of a cannula for facilitating extraction of mixed stem cell material and protective solution from the pouches 15 and 22 as will be described below.

The primary heat seals 18 of the heat seal areas 19 terminate short of the opposite side edge 5b, and define with the peripheral seal 4 along the side edge 5b an elongated manifold 25 which communicates the respective pouches 15 with the inlet port 9 through the mixing region 16.

In general, this is the form in which the container 1 is supplied, and is supplied sterile ready to receive the stem cell material and the protective solution through the inlet port 9. The closure plug 14 seals the container 1, thereby maintaining the hollow interior region 8 of the container 1 sterile. Initially the inlet tube 13 is severed for severing the closure plug 14 therefrom, and is immediately secured to a supply tube 28 by heat welding at 29 in order to maintain the hollow interior region 8 of the container 1 sterile. The supply tube 28 is coupled to a stem cell supply source illustrated by broken lines 30, which typically is a bag of a flexible plastics material. An appropriate quantity of the stem cell material is delivered into the mixing region 16 from the supply source 30. The supply tube 28 is next coupled to a supply of the protective solution, and an appropriate volume of the protective solution is delivered into the mixing region 16 through the inlet port 9 for mixing with the stem cell material in the mixing region 16.

On the stem cell material and the protective solution being homogenously mixed, the panels 3 are secured together by two further primary heat seals 34 along spaced apart primary heat seal areas 35 to form the two additional pouches 22 in the mixing region 16, which are similar to the pouches 15. The primary heat seals 34 and the primary heat seal areas 35 are similar to the primary heat seals 18 and the primary heat seal areas 19, and are of width sufficient to permit subsequent severing of the additional pouches 22 along cut lines 36 without affecting the integrity of the primary heat seals 34. The primary heat seals 34 terminate short of the portion of the peripheral seal 4 which extends along the side edge 5b, so that the manifold 25 communicates with the two pouches 22. The portion of the peripheral seal 4 which extends along the end edge 6b of the container 1 defines with the nearest-most primary heat seal 34 a communicating chamber 38 which communicates the manifold 25 with the inlet port 9.

Once the primary heat seals 34 have been formed, the mixed stem cell material and the protective solution is urged along the manifold 25 and into the respective pouches 15 and 22, so that each pouch 15 and 22 contains a substantially similar quantity of the mixed stem cell material and protective solution to that of the other pouches 15 and 22. The panels 3 are then heat sealed along an elongated secondary heat seal 39 extending along a secondary heat seal area 40 which extends transversely of the pouches 15 and 22 and parallel to the portion of the peripheral seal 4 which extends along the side edge 5b. The secondary heat seal 32 co-operates with the primary heat seals 18 and 34 and the portion of the peripheral seal 4 adjacent the end edge 6a to isolate the respective pouches 15 and 22 from the manifold 25 and from each other, to thereby seal the mixed stem cell material and the protective solution within the respective pouches 15 and 22.

The pouches 15 and 22 are then severed from the container 1 along the transverse cut lines 17 and 36 which extend through the primary heat seal areas 19 and 35, and along a longitudinal cut line 42 which extends along the secondary heat seal area 40 between the secondary heat seal area 40 and the portion of the peripheral seal 4 which extends along the side edge 5b of the panels 3. The pouches 15 and 22 are then placed in discrete envelopes (not shown), and are loaded into the cryogenic apparatus (also not shown) for freezing therein. The envelopes contain identifying data, which identifies the animal from which the stem cells were obtained and other relevant data, such as the date on which the stem cells were obtained and the source from which the stem cells came, as well as the date of freezing.

When it is desired to treat the horse with the stem cell material, one or more of the pouches 15 and 22 are removed from the cryogenic apparatus. A syringe (not shown) having a pointed cannula (also not shown) extending therefrom is used for extracting the mixed stem cell material and protective solution from each pouch 15 or 22 through the outlet port 20. The membrane 24 of the outlet plug 21 of the pouch 15 or 22 is punctured by the pointed tip of the cannula, and the mixed stem cell material and protective solution is withdrawn from the pouch 15 or 22. Thereafter the animal is appropriately treated with the stem cell material.

Referring now to FIG. 9, there is illustrated a container according to another embodiment of the invention, indicated generally by the reference numeral 60, for cryogenically storing a flowable bodily material of a human or animal, which in this embodiment of the invention may also be stem cell material, of the type described with reference to the container of FIGS. 1 to 8. The container 60 is substantially similar to the container 1, and similar components are identified by the same reference numerals. In this embodiment of the invention the container 60 is supplied with an original peripheral seal 61 which extends completely around the periphery of the panels 3 from which the container 60 is formed. However, in this embodiment of the invention the panels 3 are considerably wider between the opposite side edges 5a and 5b than the panels of the container 1. This permits the formation of discrete pouches 15 of greater length than can be formed in the container 1.

However, in this embodiment of the invention the peripheral seal 4 which defines with the panels 3 the hollow interior region 8 is formed by an additional heat seal 63 extending between the original peripheral seal 61 which extends along the opposite end edges 6a and 6b, and is located intermediate the side edges 5a and 5b. In this embodiment of the invention the additional heat seal 63 defines the hollow interior region 8 with the remainder of the original peripheral seal 61 extending along the side edge 5a and extending along the end edges 6a and 6b between the side edge 5a and the additional peripheral seal 63.

Additionally, in this embodiment of the invention the primary heat seal area 19a which is closest to the inlet port 9 extends from the peripheral seal 4 adjacent the side edge 5a at an angle less than 90°, and in a general direction towards the adjacent primary heat seal 19. In this embodiment of the invention the manifold 25 forms the mixing region within which the stem cell material and the protective solution are mixed.

It has been found that by providing the primary heat seal area 19a to extend from the peripheral seal 4 adjacent the side edge 5a at an angle of less than 90°, and in a general direction towards the adjacent primary heat seal area 19, the stem cell material and the protective solution can be more easily delivered into the manifold 25 for mixing thereof, and air pockets can be more easily dislodged from the stem cell material and the protective solution, both prior to and during mixing of the stem cell material with the protective solution and also subsequent to mixing. Additionally, the angled primary heat seal area 19a also enhances fluid flow characteristic in the container, thereby optimising distribution of the mixed stem cell material and protective solution to the discrete pouches 15.

Ideally, in this embodiment of the invention the mixing of the stem cell material and the protective solution in the manifold 25 is carried out with the container 1 extending in a vertical plane with the manifold 25 at the lower end of the container. On completion of mixing, the container 1 is then inverted, and the mixed stem cell material and the protective solution flows into the pouches 15. The panels 3 are then heat sealed along a secondary heat seal (not shown) which is similar to the secondary heat seal 39 of the container 1. The secondary heat seal (not shown) co-operates with the primary heat seals 18 to thereby seal and isolate the pouches 15 from the manifold 25, and in turn from each other.

Referring now to FIG. 10 there is illustrated a container according to another embodiment of the invention indicated generally by the reference numeral 70, for cryogenically storing a flowable bodily material of a human or animal, which in this embodiment of the invention may also be stem cell material, of the type described with reference to the container of FIGS. 1 to 8. The container 70 is substantially similar to the container 60, and similar components are identified by the same reference numerals. The only difference between the container 70 and the container 60 is that a second inlet port 71 is provided to the container 70 for accommodating the DMSO saline solution into the container where it is desired to introduce the DMSO saline solution into the container independently of the stem cell material which is introduced to the container through the inlet port 9. A sub-micron filter 72 is located in the inlet port 71 for filtering the DMSO saline solution as it is passing through the inlet port 71. An advantage of the container 70 is that it facilitates simultaneous charging of the container 70 with the stem cell material and the DMSO saline solution. Simultaneous charging of the container 70 with the stem cell material and the DMSO saline solution, tends to assist in mixing of the two materials.

Otherwise the container 70 and its use is similar to that of the container 60 described with reference to FIG. 9.

While the containers 1, 60 and 70 have been described as being supplied with the panels 3 secured together by the peripheral seal 4 and the primary seals 18 to form the five pouches 15 and the mixing region 16, and with the inlet and outlet ports 9 and 20 secured between the panels 3, it is envisaged that the containers 1, 60 and 70 may be supplied with the panels secured only with the peripheral seal 4, and with the inlet and outlet ports 9 and 20 sealably secured in the peripheral seal 4. In which case, the primary seals could be formed subsequently.

While the containers 1, 60 and 70 have been described with the outlet ports 20 sealably located between the panels 3, it is envisaged that in certain cases, the container 1 may be supplied without the outlet ports 20 being secured to the containers 1, 60 and 70, and in which case, it is envisaged that the containers 1, 60 and 70 would be supplied with the side edges 5a of the panels 3 unsealed, and the outlet ports 20 could be subsequently secured between the panels 3 along the side edge 5a during forming of the peripheral seal 4 along the side edge 5a. Similarly, the containers 1, 60 and 70 may be supplied without the one or both inlet ports, and in which case, the container would be provided without the portion of the peripheral seal which extends along the end edge 6b, and the one or both inlet ports, as the case may be, would then be heat sealed to the panels when forming the peripheral seal along the end edge 6b.

While the containers 1, 60 and 70 have been described for storing stem cell material, the containers 1, 60 and 70 may be used for storing any other type of material, and also, may be used for storing any type of flowable material, whether the material is to be subjected to cryogenic temperatures or otherwise.

While the containers have been described for storing stem cell material harvested from an umbilical cord and a placenta after birth of a foal, the containers may be used for storing stem cell material from any type of human or animal.

While the containers have been described as being of FEP plastics material, the containers may be of any other suitable heat sealable material, and while the material of the containers has been described as being transparent, while this is advantageous, it is not essential.

While the containers 60 and 70 have been described as having an additional heat seal 63, the heat seal 63 could be omitted, and in which case the hollow interior regions of the containers 60 and 70 would be defined by the peripheral seal 61.

1-77. (canceled)

78. A method for storing a flowable bodily material from a human or animal body, the method comprising providing a container comprising a pair of panels of flexible material sealed together to define a hollow interior region by a peripheral seal extending around the hollow interior region, and an inlet port being provided to the hollow interior region, securing the panels together to define at least two discrete pouches and a manifold in the hollow interior region with the manifold communicating the respective pouches with the inlet port, delivering the bodily material through the inlet port to the pouches through the main manifold, and further securing the panels together to isolate the pouches from the manifold and from each other with the bodily material sealed therein.

79. A method as claimed in claim 78 in which the panels are secured together to define a mixing region in the hollow interior region for mixing the flowable bodily material with a protective material, and preferably, the mixing region is located adjacent the inlet port, and advantageously, the manifold forms the mixing region, and alternatively, the manifold communicates with the inlet port through the mixing region.

80. A method as claimed in claim 79 in which the panels are secured together at a location in the mixing region to form at least one additional discrete pouch from the mixing region, the at least one additional discrete pouch communicating with the manifold, and preferably, the bodily material and the protective material are mixed in the mixing region prior to form the at least one additional pouch in the mixing region.

81. A method as claimed in claim 78 in which the panels are secured together along respective spaced apart elongated primary sealing areas to define the discrete pouches, and preferably, the primary sealing areas with the exception of the primary sealing area closest to the inlet port extend substantially parallel to each other, and advantageously, the primary sealing area which is closest to the inlet port extends in a general direction towards the adjacent primary sealing area, and preferably, the primary sealing area which forms the at least one additional discrete pouch in the mixing region which is closest to the inlet port extends in a general direction towards the adjacent primary sealing area, and advantageously, the primary sealing areas extend from the peripheral seal, and preferably, the primary sealing areas extend from the peripheral seal substantially perpendicularly thereto, and advantageously, the panels are secured together along the primary sealing areas by heat sealing the panels, and preferably, the primary sealing areas are of sufficient width to permit severing of the sealed pouches from the container along the primary sealing areas without affecting the integrity of the seals.

82. A method as claimed in claim 78 in which the panels are secured together along secondary sealing areas for isolating the respective pouches from the manifold and from each other to sealably close the respective pouches, and preferably, the panels are secured together along the secondary sealing area by heat sealing the panels, and preferably, the secondary sealing area is of sufficient width to permit severing of the sealed pouches from the container along the secondary sealing area without affecting the integrity of the secondary seal, and advantageously, the pouches extend side by side from the peripheral seal, and preferably, the manifold extends transversely relative to the pouches.

83. A method as claimed in claim 78 in which a sealed outlet port is provided from each pouch, and preferably, each sealed outlet port is adapted to be punctured for withdrawing the bodily material therefrom, and advantageously, each sealed outlet port is adapted to be punctured by a pointed tip of a cannula, and preferably, the sealed outlet ports are located adjacent one end of the respective pouches, and advantageously, the sealed outlet ports are located adjacent a portion of the peripheral seal which extends adjacent the edge of the panels, and preferably, each sealed outlet port is located between the panels adjacent the peripheral seal and the panels are sealably secured to the sealed outlet ports, and advantageously, the panels are sealably secured to the outlet ports by heat sealing.

84. A method as claimed in claim 78 in which the peripheral seal which defines the hollow interior region between the panels extends around the periphery of the panels, and alternatively, the peripheral seal which defines the hollow interior region between the panels is spaced apart from a portion of the periphery of the panels.

85. A method as claimed in claim 78 in which a second inlet port is provided to the hollow interior region for accommodating a protective material for mixing with the bodily material, and preferably, each inlet port is located between the panels adjacent the peripheral seal, and is sealably secured to the panel, and preferably, each inlet port is sealably secured to the panels by heat sealing, and advantageously, each inlet port is adapted to be sealably coupled to a corresponding supply tube from which the bodily material is supplied to the inlet port, and preferably, an inlet tube extends outwardly from each inlet port and is adapted for sealably coupling to the corresponding supply tube, and advantageously, the inlet tube of each inlet port is adapted to be sealably coupled to the corresponding supply tube by heat sealing, and preferably, the container is adapted for storing stem cell material extracted from blood.

86. A container for use in the method as claimed in claim 76.

87. A container for storing a flowable bodily material of a human or animal, the container comprising a pair of panels of flexible material sealed together by a peripheral seal to define a hollow interior region, and an inlet port being provided to the hollow interior region, the panels being one of secured together to define at least two discrete pouches and a manifold and adapted to be secured together to define the at least two discrete pouches and the manifold in the hollow interior region with the manifold communicating the respective pouches with the inlet port.

88. A container as claimed in claim 87 in which the panels are one of secured together to define a mixing region and adapted to be secured together to define the mixing region in the hollow interior region for mixing the flowable bodily material with a protective material, and preferably, the mixing region is located adjacent the inlet port.

89. A container as claimed in claim 88 in which the manifold forms the mixing region, and preferably, the manifold communicates with the inlet port through the mixing region.

90. A container as claimed in claim 88 in which the panels are adapted to be secured together at a location within the mixing region to form at least one additional discrete pouch in the mixing region communicating with the manifold.

91. A container as claimed in claim 78 in which each pouch is an elongated pouch, and preferably, the pouches extend side by side from the peripheral seal.

92. A container as claimed in claims 78 in which the manifold extends transversely relative to the pouches.

93. A container as claimed in claim 78 in which the panels are secured together along respective spaced apart elongated primary sealing areas to define the discrete pouches, and preferably, the primary sealing areas extend parallel to each other with the exception of the primary sealing area located closest to the inlet port, and advantageously, the primary sealing area which is located closest to the inlet port extends in a general direction towards the adjacent primary sealing area, and preferably, the primary sealing area which separates the mixing region from the adjacent discrete pouch extends in a general direction towards the adjacent primary sealing area, and advantageously, the primary sealing area which forms the at least one additional discrete pouch in the mixing region, and which is closest to the inlet port extends in a general direction towards the adjacent primary sealing area, and preferably, the primary sealing areas extend from the peripheral seal, and preferably, the primary sealing areas extend from the peripheral seal substantially perpendicularly thereto, and advantageously, the primary sealing areas are of sufficient width to permit severing of the sealed pouches from the container without affecting the integrity of the seals.

94. A container as claimed in claim 78 in which the panels are adapted to be secured together along secondary sealing areas for isolating the respective pouches from the manifold and from each other to sealably close the respective pouches, and preferably, the secondary sealing area is of sufficient width to permit severing of the sealed pouches from the container along the secondary sealing area without affecting the integrity of the secondary seal, and advantageously, the peripheral seal which defines the hollow interior region between the panels extends around the periphery of the panels, and alternatively, the peripheral seal which defines the hollow interior region between the panels is spaced apart from a portion of an edge of the panels.

95. A container as claimed in claim 78 in which the container is one of provided with a plurality of sealed outlet ports and adapted to be provided with the plurality of sealed outlet ports, such that one of the sealed outlet ports is provided from each pouch, and preferably, each sealed outlet port is adapted to be punctured for withdrawing the bodily material from the pouch, and advantageously, each outlet port is adapted to be punctured by a pointed tip of a cannula, and preferably, the sealed outlet ports are located adjacent one end of the respective pouches, and advantageously, the outlet ports are located between the panels adjacent the peripheral seal, and preferably, the panels are sealably secured to the outlet ports, and advantageously, the panels are sealably secured to the outlet ports by heat sealing.

96. A container as claimed in claim 78 in which a second inlet port is provided to the hollow interior region for accommodating the protective material for mixing with the bodily material, and preferably, each inlet port is located adjacent the peripheral seal, and preferably, each inlet port is located between the panels, and advantageously, the panels are sealably secured to each inlet port, and preferably, the panels are sealably secured to each inlet port by heat sealing, and advantageously, each inlet port is adapted to be sealably coupled to a corresponding supply tube, and preferably, an inlet tube is sealably secured to each inlet port and extends outwardly therefrom, the inlet tube of each inlet port being adapted to be sealably coupled to the corresponding supply tube, and advantageously, the inlet tube of each inlet port is adapted to be sealably coupled to the corresponding supply tube by heat sealing.

97. A container as claimed in claim 78 in which the material of the panels of the container is a material resistant to cryogenic temperatures, and preferably, the material of the panels of the container is of a material which does not react with the bodily material, and advantageously, the material of the panels of the container is a fluorinated ethylene-propylene (FEP) plastics material, and preferably, the container is adapted for storing stem cell material extracted from blood.
